(19) United States
(12) Patent Application Publication
(10) Pub. No.: US 2008/0241190 A1
(43) Pub. Date: Oct. 2, 2008

(54) VACCINATION OF HORSES AGAINST LAWSONIA INTRACELLULARIS

(75) Inventors: Jeremy Kroll, Ames, IA (US); Axel Neubauer, Saint Joseph, MO (US); Philip Utley, Ames, IA (US); Michael Roof, Ames, IA (US); Juergen Daemmgen, Ochsenhausen (DE); Knut Elbers, Gau Algesheim (DE)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: BOEHRINGER INGELHEIM VETMEDICA, INC., Saint Joseph, MO (US)

(21) Appl. No.: 11/937,657

(22) Filed: Nov. 9, 2007

Related U.S. Application Data
(60) Provisional application No. 60/865,547, filed on Nov. 13, 2006.

(51) Int. Cl. A61K 39/02; A61P 31/04

(52) U.S. Cl. 424/234.1

(57) ABSTRACT

The present invention is broadly concerned with vaccination of horses against proliferative enteritis, preferably equine proliferative ileitis, which is caused by an obligate intracellular bacterium Lawsonia Intracellularis (L. intracellularis). Specifically, the invention provides for a method of providing immune protection against L. intracellularis by vaccinating horses, preferably foals starting from one (1) week of age. Preferably the foals are vaccinated with about 4.9 log 10 to about 6.9 log 10 of a live modified L. intracellularis bacteria per dose.

### BACKGROUND OF THE INVENTION

1. Field of the Invention

The present invention is broadly concerned with vaccination of horses against proliferative enteritis, known as ileitis, preferably equine proliferative ileitis, which is caused by an obligate intracellular bacterium Lawsonia Intracellularis (L. intracellularis). Specifically, the invention provides for a method of providing immune protection against L. intracellularis by vaccinating horses, preferably foals starting from one (1) week of age against L. intracellularis bacteria Preferably the foals are vaccinated with about 4.9 log 10 to about 6.9 log 10 of a live modified L. intracellularis bacteria per dose.

2. Description of the Prior Art

Proliferative enteritis (PE) caused by L. intracellularis is a disease of high economic impact in swine worldwide. L. intracellularis is an obligate, intracellular bacterium, described for example in S. McOrist et al., Infection and Immunity, Vol. 61, No. 19, 4286-4292 (1993) and G. Lawson et al., J. of Clinical Microbiologic, Vol. 31, No. 5, 1136-1142 (1993).

The disease was first identified in swine and characterized by its gross and microscopic pathology, and later by the demonstration of the intracellular bacteria within affected cells. The characterizing pathological feature of the disease is the proliferation of immature epithelial cells in the crypts of the ileum (terminal part of the small intestine), the large intestine or both. Sections of infected tissue are characterized by a reddened thickening mucosa resembling a “garden hose,” and enteric lesions. The gut thickening ultimately prevents normal gut function, absorption capabilities, and nutrient transfer. Clinical effects of the disease are chronic weight loss, unthriftiness, diarrhea, and death. The disease is of economic importance owing to death loss, increased medication costs, poor weight gain and decreased food conversion in affected animals. Clinical cases of ileitis are observed and most notably in pigs 6-20 weeks of age. However, the presence of L. intracellularis has been confirmed by polymerase chain reaction (PCR) in recently weaned pigs (3-4 weeks of age), suggesting subclinical L. intracellularis exposure occur in the nursery and perhaps, originates from Lawsonia-positive dams (Mauch and Bilkei (2004) Vet Rec 155: 532; Marsteller et al. (2003). Swine Health Prod 11:127-130; Stege et al. (2004) Vet Micro 104: 197-206).

Sporadic infection with L. intracellularis has been described in many other mammalian and avian species including dogs, hamsters, rabbits, blues foxes macaques and ostriches. However, there are few case reports of L. intracellularis infections in horses and most have been restricted to North America (Williams et al., (1996) J. Vet. Diagn. Invest. 8, 254-256; Lavoie et al., (2000) Equine VeL J. 32, 418-425). Recently, the first case has been reported in Australia (McClintock and Collins (2004) Aust. Vet. J. 82, 750-752). In Switzerland, the prevalence of L. intracellularis in pig herds is rapidly increasing because of restricted use of tetracycline and changes in group size and management. In addition clinical cases of equine PE are also reported in Switzerland by Wuersch et al., (2006) J. Vet. Med. 53, 17-21.

Equine PE caused by L. intracellularis has recently been described as an intestinal disease clinically characterized by weight loss, fever, diarrhea, colic, leucocytosis and hypoproteinaemia. However, clinical signs in equine PE are unspecific and render the clinical diagnosis challenging. Hypoproteinaemia, in this case is most probably due to malabsorption and protein loss into the intestinal lumen, is described as the most consistent clinical finding and is suggested as a screening parameter for suspected cases of equine PE. Increased serum CK, AST and LDH of the filly indicate muscle damage, whereas increased CK concentrations have been described previously with equine PE and were attributed to the catabolic state of the affected animals (Lavoie et al., (2000) Equine Vet. J. 32, 418-425).

Like pigs, horses seem to be most susceptible to infection shortly after weaning. In one study the affected age group ranged from 3 to 13 months (Lavoie et al., (2000) Equine Vet. J. 32, 418-425). These findings are supported by other case reports (Williams et al., (1996). J. Vet. Diagn. Invest. 8, 254-256; Frank, et al., (1998) Equine Vet. J. 30, 549-552; Brees et al., (1999) J. Am. Vet. Med. Assoc. 215, 511-514; McClintock and Collins (2004) Aust. Vet. J. 82, 750-752).

Current vaccination strategies for the prevention treatment of proliferative enteritis are limited to swine. For example, U.S. Pat. Nos. 5,714,375 and 5,885,823 as well as WO 05/011731, all of which are herein incorporated by reference in their entireties, provides vaccines for the immunization of swine. Those vaccines are highly effective and known in the market as Enterisol®Ileitis or Enterisol®Ileitis B3903 (Boehringer Ingelheim Vetmedica Inc., St Joseph, Mo., USA).

Yet no vaccination strategy or vaccine exists for the prevention or treatment of horses, in particular of foals. Prior to the method of the present invention, proliferative enteritis in horses was either not diagnosed, or treated conservative after onset of infection with antibiotics. So far, no method of prevention of L. intracellularis infections exists.

Thus, there was a long lasting need for the prevention or prophylactic treatment of horses, preferably foals, against proliferative enteritis caused by L. intracellularis bacteria.

### DESCRIPTION OF THE INVENTION

The present invention relates to a method for prevention or treatment of a horse against L. intracellularis infections or for reduction of clinical symptoms caused by L. intracellularis in a horse, comprising the step administering to said horse an effective dose of L. intracellularis antigen.

The term “prevention” or “treatment” as used herein means, but is not limited to a process which includes the administration of an L. intracellularis antigen to an animal, wherein said L. intracellularis antigen, when administered to said animal elicits or is able to elicit an immune response in said animal against L. intracellularis.

The term “clinical symptoms” refers to any abnormal clinical manifestation in an animal as compared to an healthy animal. Preferably, clinical symptoms according to the invention are chronic weight loss, unthriftiness, fever, diarrhea, colic, leucocytosis and/or hypoproteinaemia.

Thus, the term “reduction of clinical symptoms” shall mean, but not limited to the reduction of any of the clinical symptoms listed herein. Howsoever, it should also refer to any reduction of pathogen shedding, reduction in transmission of L. intracellularis and reduction of antibiotic treatment necessity.

The term “effective dose” as used herein means, but is not limited to an amount of antigen, that elicits or is able to elicits an immune response in an animal, to which said effective dose of L. intracellularis antigen is administered.

An “immunological or immune response” to a composition or vaccine shall mean the development in the host of a cellular and/or antibody-mediated immune response to the composition or vaccine of interest. Thus, the term “elicits or is able to elicit an immune response” means, but is not limited to an immunological process in a host characterized in that said host develops a cellular and/or antibody-mediated immune response to the composition or vaccine of interest. Usually, an “immune response” includes but is not limited to one or more of the following effects: The production or activation of antibodies, B cells, helper T cells, suppressor T cells, and/or cytotoxic T cells and/or yd T cells, directed specifically to an antigen or antigens included in the composition or vaccine of interest. Preferably, the host will displays either a therapeutic or protective immunological response such that resistance to new infection will be enhanced and/or the clinical severity of the disease reduced. Such protection will be demonstrated by either a reduction or lack of the symptoms associated with host infections as described above.

The amount of antigen that is effective to elicit an immune response or is able to elicit an immune response in an animal depends on the ingredients of the vaccine and the schedule of administration.

Typically, when killed bacterial antigen is used in the vaccine, the vaccine contains an amount of about 10⁵ to about 10⁹ colony forming units (CFU) of the bacterium per dose, preferably, about 10⁶ to about 10⁸ (CFU) of the bacterium per dose.

In particular, when modified live L. intracellularis bacteria are used in the vaccines, e.g. the bacteria isolates designated isolate B3903, ATCC accession No. PTA4926 and designated isolate N34NP40wk, ATCC accession No. 55783 (both described in WO 96/39629 and WO 05/011731), the recommended dose to be administered to the susceptible animal is preferably about 4.5 log 10 TCID₅₀ (tissue culture infective dose 50% end point)/dose to about 9.0 log 10 TCID₅₀/dose and more preferably about 4.9 log 10 TCID₅₀/dose to about 6.9 log 10 TCID₅₀/dose. In a preferred embodiment, the titer of the vaccine is about 5.9 log 10 TCID₅₀/dose as determined by Tissue Culture Infective Dose 50% endpoint dilution assay (TCID₅₀). In general, the quantity of immunogen will be between 50 and 5000 micrograms, and between 4.5 log 10 and 9.0 log 10 TCID₅₀, more preferably between 4.9 log 10 and 6.9 log 10 TCID₅₀, when purified bacteria are used.

Sub-unit vaccines are normally administered with an antigen inclusion level of at least 2 μg antigen per dose, preferably with about 2 to about 500 μg/dose, still more preferably with about 5 to about 400 μg/dose, even more preferably with about 8 to about 300 μg/dose, still more preferably with about 10 to about 200 μg/dose, still more preferably with about 10 to about 150 μg/dose, still more preferably with about 10 to about 100 μg/dose, still more preferably with about 10 to about 75 μg/dose, still more preferably with about 10 to about 50 μg/dose, still more preferably with about 10 to about 20 μg/dose. Administration preferably occurs via parenteral route such as intra muscularly or subcutaneously for example.

As used herein the term “reduction” means, but is not limited to a statistically significant reduction of one or more clinical symptoms which are associated with L. intracellularis infections (frequency of cross lesions, etc.) in a vaccinated group of animals vs. a non-vaccinated control group of animals. The term “statistically significant reduction of clinical symptoms” means but is not limited to, that the frequency in the incidence of at least one clinical symptom in the vaccinated group of animals is at least 20%, preferably 30%, even more preferably 50%, most preferably 70% lower than in the non-vaccinated control group after the challenge with an infectious L. intracellularis bacteria.

As used herein, the term “L. intracellularis” means the intracellular, curved gram-negative bacteria described in detail by C. Gebhart et al., Int'l. J. of Systemic Bacteriology, Vol. 43, No. 3, 533-538 (1993) and S. McOrist et al., Int'l. J. of Systemic Bacteriology, Vol. 45, No. 4, 820-825 (1995), each of which is incorporated herein by reference in their entireties, and includes but is not limited to the isolates described in WO 96/39629 and WO 05/011731. In particular, the term “L. intracellularis” also means, but is not limited to the isolates deposited under the Budapest Treaty with the American Type Culture Collection, 10801 University Boulevard, Manassas, Va. 20110-2209 and assigned ATCC accession number PTA 4926 or ATCC accession number 55783. Both isolates are described in WO 96/39629 and WO 05/011731, respectively. The term “L. intracellularis” also means, but is not limited to any other L. intracellularis bacteria strain, or isolate, preferably having the immunogenic properties of at least one of the L. intracellularis strains described in WO 96/39629 and WO 05/011731, in particular having the immunogenic properties of at least one of the isolates deposited under the Budapest Treaty with the American Type Culture Collection, 10801 University Boulevard, Manassas, Va. 20110-2209 and assigned ATCC accession numbers PTA 4926 or ATCC accession number 55783.

A strain or isolate has the “immunogenic properties” of at least one of the L. intracellularis strains described in WO 96/39629 and WO 05/011731, in particular, of the isolates deposited as ATCC accession numbers PTA 4926 or ATCC accession number 55783, when it is detectable at least with one of the anti-L. intracellularis specific antibodies, described in WO06/01294, in an detection assay that is also described in WO06/01294. Preferably those antibodies are selected from the antibodies having the reference numbers 301:39, 287:6, 268:29, 110:9, 113:2 and 268:18. Preferably, the detection assay is an sandwich ELISA as described in Examples 2 and 3 of WO06/12949, whereas antibody 110:9 is used as an capture antibody and antibody 268:29 is used as conjugated antibody. All antibodies disclosed in WO06/12949 are produced by hybridoma cells, which are deposited at the Centre for Applied Microbiology and Research (CAMR) and European Collection of Cell Cultures (ECACC)”, Salisbuiy, Wiltshire SP4 0JG, UK, as patent deposit according to the Budapest Treaty. The date of deposit was May 11, 2004. HYBRIDOMA CELL LINE 110:9 is successfully deposited under ECACC Acc. No. 04092204. HYBRIDOMA CELL LINE 113:2 is successfully deposited under ECACC Acc. No. 04092201. HYBRIDOMA CELL LINE 268:18 is successfully deposited under ECACC Acc. No. 04092202. HYBRIDOMA CELL LINE 268:29 is successfully deposited under ECACC Acc. No. 04092206. HYBRIDOMA CELL LINE 287:6 is successfully deposited under ECACC Acc. No. 04092203. HYBRIDOMA CELL LINE 301:39 is successfully deposited under ECACC Acc. No. 04092205.

The term “L. intracellularis antigen” as used herein means, but is not limited to any composition of matter, that comprises at least one antigen that can induce, stimulate or enhance the immune response against a L. intracellularis-caused infection, when administered to an animal. Preferably, said L. intracellularis antigen is a complete L. intracellularis bacterium, in particular in an inactivated form (a so called killed bacterium). a modified live or attenuated L. intracellularis bacterium (a so called MLB), any sub-unit, polypeptide or component of L. intracellularis, or any chimeric vector each comprises at least an immunogenic amino acid sequence of L. intracellularis. The terms “immunogenic protein”, “immunogenic polypeptide” or “immunogenic amino acid sequence” as used herein refer to any amino acid sequence which elicits an immune response in a host against a pathogen comprising said immunogenic protein, immunogenic polypeptide or immunogenic amino acid sequence. In particular, an “immunogenic protein”, “immunogenic polypeptide” or “immunogenic amino acid sequence” of L. intracellularis means any amino acid sequence that codes for an antigen which elicits an immunological response against L. intracellularis in a host to which said “immunogenic protein”, “immunogenic polypeptide” or “immunogenic amino acid sequence” is administered.

An “immunogenic protein”, “immunogenic polypeptide” or “immunogenic amino acid sequence” as used herein, includes but is not limited to the full-length sequence of any proteins, analogs thereof, or immunogenic fragments thereof. The term “immunogenic fragment” means a fragment of a protein which includes one or more epitopes and thus elicits the immunological response against the relevant pathogen. Such fragments can be identified using any number of epitope mapping techniques that are well known in the art. See, e.g., Epitope Mapping Protocols in Methods in Molecular Biology, Vol. 66 (Glenn E. Morris, Ed., 1996) Humana Press, Totowa, N.J. (The teachings and content of which are incorporated by reference herein.) For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715. (The teachings and content of which are incorporated by reference herein.) Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Synthetic antigens are also included within the definition, for example, polyepitopes, flanking epitopes, and other recombinant or synthetically derived antigens. See, e.g., Bergmann et al. (1993) Eur. J. Immunol. 23:2777-2781; Bergmann et al. (1996), J. Immunol. 157:3242-3249; Suhrbier, A. (1997), Immunol and Cell Biol. 75:402-408; Gardner et al., (1998) 12th World AIDS Conference, Geneva, Switzerland, Jun. 28-Jul. 3, 1998. (The teachings and content of which are incorporated by reference herein.)

Suitable L. intracellularis antigens include, but are not limited to those described in EP 1219711; U.S. Pat. No. 6,605,696; WO 96/39629; WO97/20050; WO 00/69903; WO 00/69904; WO 00/69905; WO 00/69906; WO 02/38594; WO 02/26250; WO 03/006665; WO 04/033631; WO 05/026200; WO 05/011731; WO 06/113782 and WO 06/099561.

According to a further aspect, the present invention relates to a method for prevention or treatment of a foal, preferably of one (1) week of age or older against L. intracellularis infections or for reduction of clinical symptoms caused by L. intracellularis in such foals. Thus according to a further aspect, the present invention relates to a method for prevention or treatment of a horse against L. intracellularis infections or for reduction of clinical symptoms caused by L. intracellularis in a horse, comprising the step administering to a horse of (1) week of age or older an effective dose of L. intracellularis antigen.

As clinical manifestations of equine PE seem to occur between months 3 and months 13 of age, horses should be treated with an effective dose of L. intracellularis antigen between week one (1) and twelve (12) of age, preferably week one (1) and ten (10) of age, even more preferably week one (1) and eight (8) of age, even more preferably between week one (1) and seven (7) of age, even more preferably between week one (1) and six (6) of age, even more preferably between week one (1) and five (5) of age, and most preferably between week one (1) and four (4) of age.

In view of interferences with maternal anti-L. intracellularis antibodies, vaccination or treatment might not be done before week four (4), preferably week six (6), even more preferably week seven (7) or eight (8) of age. However, the presence and interference of maternal anti-L. intracellularis antibodies can be tested and evaluated by a person skilled in the art according to standard procedures. Thus, according to a further aspect, the present invention also relates to a method for prevention or treatment of a horse against L. intracellularis infections or for reduction of clinical symptoms caused by L. intracellularis in a horse, comprising the step administering to a horse older than (4) weeks of age, preferably older than five (5) week of age, even more preferably older than six (6), even more preferably older than seven (7), more preferably older than eight (8) weeks of age in cause of the presence of anti-L. intracellularis antibodies. However, most preferably vaccination/treatment should be done in foals not older than twelve (12) weeks of age whenever possible. Thus, according to a further aspect, the present invention also relates to a method for prevention or treatment of a horse against L. intracellularis infections or for reduction of clinical symptoms caused by L. intracellularis in a horse, comprising the step administering to a horse not older than twelve (12) weeks of age but older than (4) weeks of age, preferably but older than five (5) week of age, even more preferably but older than six (6), even more preferably but older than seven (7), most preferably but older than eight (8) weeks of age in cause of the presence of anti-L. intracellularis antibodies. If foals are not vaccinated, vaccination of older horses is also possible and within the meaning of the present invention. The aspect of interference with maternal anti-L. intracellularis antibodies and is effect on the prevention/treatment of horses/foals with L. intracellularis antigen should be considered for every embodiment of the invention as described herein.

According to a further aspect, to a method for prevention or treatment of a horse against L. intracellularis infections or for reduction of clinical symptoms caused by L. intracellularis in a horse, comprising the step administering to a horse of one (1) week of age or older, preferably between week (1) and twelve (12) of age an effective dose of L. intracellularis antigen, wherein the L. intracellularis antigen is selected from the group consisting of live modified L. intracellularis bacteria, killed L. intracellularis bacteria or one or more sub-units of L. intracellularis bacteria. Preferably, the vaccine comprises modified live L. intracellularis bacteria. More preferably, the vaccine is Enterisol® Ileitis or Enterisol® Ileitis B3903 (Boehringer Ingelheim Vetmedica, Inc.).

According to a further aspect, the present invention relates to a method for prevention or treatment of a horse against L. intracellularis infections or for reduction of clinical symptoms caused by L. intracellularis in a horse, comprising the step administering to said horse at week (1) of age or older an a dose of about 4.5 log 10 TCID₅₀ to about 6.9 log 10 TCID₅₀ of the live modified L. intracellularis bacteria. Preferably, said life modified L. intracellularis bacteria are those included in the vaccine Enterisol® Ileitis or Enterisol® Ileitis B3903 (Boehringer Ingelheim Vetmedica, Inc.). Preferably said L. intracellularis antigen is the vaccine Enterisol® Ileitis or Enterisol® Ileitis B3903 (Boehringer Ingelheim Vetmedica, Inc.). As mentioned above, preferably said vaccination/treatment occurs between week one (1) and twelve (12) of age, preferably week one (1) and ten (10) of age, even more preferably between week one (1) and eight (8) of age, even more preferably between week one (1) and seven (7) of age, even more preferably between week one (1) and six (6) of age, even more preferably between week one (1) and five (5) of age, and most preferably between week one (1) and four (4) of age. Preferably, said live modified L. intracellularis bacteria are administered orally.

It is hereby understood that the treatment of horses, preferably foals with L. intracellularis antigen as described herein, is also effective for the prevention of subclinical effects, preferably subclinical enteritis in horses and foals, respectively. “Subclinical effects” are characterized by clinical manifestations, which are not visible or apparent in the absence a specific diagnostic means. Subclinically infected animals may be diagnosed either by detection of L. intracellularis in the feces, serology or measurable differences in weight. Moreover, subclinical L. intracellularis infections refer to foals/horses that do not suffer from physical enteritis-associated pain but are characterized by a decrease in physical development such as weight gain, reduced body size as well as development of optimal skeletal muscle mass and body shape. These specific subclinical manifestations can be due to a reduction in nutrient uptake caused by intestinal abnormalities directly associated with L. intracellularis infection. Those foals/horses will be colonized and shed L. intracellularis thus providing a source of infection for stable-mate horses.

Thus, according to a further aspect, the present invention also relates to the prevention of subclinical effects, preferably subclinical enteritis caused by or associated with a L. intracellularis infection in a horse, comprising the step administering to said horse an effective dose of L. intracellularis antigen. In view of the teaching above, the present invention also relates to the prevention of subclinical effects, preferably subclinical enteritis caused by or associated with a L. intracellularis infection in a horse, comprising the step administering to a horse of (1) week of age or older an effective dose of L. intracellularis antigen. More preferably, the present invention also relates to the prevention of subclinical effects, preferably subclinical enteritis caused by or associated with a L. intracellularis infection in a horse, comprising the step administering to a horse between week one (1) and twelve (12) of age, preferably week one (1) and ten (10) of age, even more preferably between week one (1) and eight (8) of age, even more preferably between week one (1) and seven (7) of age, even more preferably between week one (1) and six (6) of age, even more preferably between week one (1) and five (5) of age, and most preferably between week one (1) and four (4) of age an effective dose of L. intracellularis antigen. However, the aspect of interference with maternal anti-L. intracellularis antibodies should be considered and alternative prevention/treatment regimes need to be used, e.g as described supra which refers to the administration of L. intracellularis antigen between week four (4), preferably week five (5), more preferably week six (6), even more preferably week seven (7) most preferably week eight (8) and week twelve (12) of age in the presence of maternal anti-L. intracellularis antibodies that interfere with the L. intracellularis antigen. Preferably, the L. intracellularis antigen is one of those described herein and should be used in a dose as described herein. Thus, according to a further aspect the present invention also relates to the prevention of subclinical effects, preferably subclinical enteritis caused by or associated with a L. intracellularis infection in a horse, comprising the step administering to said horse at week (1) of age or older a dose of about 4.5 log 10 TCID₅₀ to about 6.9 log 10 TCID₅₀ of live modified L. intracellularis bacteria. Preferably, said life modified L. intracellularis bacteria are those included in the vaccine Enterisol® Ileitis or Enterisol® Ileitis B3903 (Boehringer Ingelheim Vetmedica, Inc.). More preferably, the vaccine is Enterisol® Ileitis or Enterisol® Ileitis B3903 (Boehringer Ingelheim Vetmedica, Inc.).

According to a further aspect, the present invention also relates to new medicinal use of an effective dose of L. intracellularis antigen for the preparation of a medicament for the prevention or treatment of a horse against L. intracellularis infections or for reduction of clinical symptoms caused by L. intracellularis in a horse, wherein said effective dose of L. intracellularis antigen is administered to a horse in of such treatment. Preferably, the treatment/vaccination is done at week one (1) of age or older. As mentioned above, preferably said vaccination occurs between week one (1) and twelve (12) of age, preferably week one (1) and ten (10) of age, even more preferably between week one (1) and eight (8) of age, even more preferably between week one (1) and seven (7) of age, even more preferably between week one (1) and six (6) of age, even more preferably between week one (1) and five (5) of age, and most preferably between week one (1) and four (4) of age. Preferably, the effective dose is that as described above, for example about 4.5 log 10 TCID₅₀ to about 6.9 log 10 TCID₅₀ when a live modified L. intracellularis bacteria is used, in particular when modified L. intracellularis bacteria is used which are included in the vaccine Enterisol® Ileitis or Enterisol® Ileitis B3903 (Boehringer Ingelheim Vetmedica, Inc.).

The manufacture of vaccine compositions comprising a L. intracellularis antigen are state of the art and known to a skilled artisan. As mentioned above, U.S. Pat. Nos. 5,714,375 and 5,885,823 as % well as WO 05/011731 describe the manufacture of several L. intracellularis comprising vaccines. Moreover, the skilled person in the art is able to knows additional components which may be comprised in said composition (see also Remington's Pharmaceutical Sciences. (1990). 18th ed. Mack Publ., Easton). The expert may use known injectable, physiologically acceptable sterile solutions. For preparing a ready-to-use solution for parenteral injection or infusion, aqueous isotonic solutions, such as e.g. saline or corresponding plasma protein solutions are readily available. The vaccine compositions may be present as lyophylisates or dry preparations, which can be reconstituted with a known injectable solution directly before use under sterile conditions, e.g. as a kit of parts.

In addition, the immunogenic and vaccine compositions of the present invention can include one or more veterinary-acceptable carriers. As used herein, “a veterinary-acceptable carrier” includes any and all solvents, dispersion media, coatings, adjuvants, stabilizing agents, diluents, preservatives, antibacterial and antifungal agents, isotonic agents, adsorption delaying agents, and the like.

“Diluents” can include water, saline, dextrose, ethanol, glycerol, and the like. Isotonic agents can include sodium chloride, dextrose, mannitol, sorbitol, and lactose, among others. Stabilizers include albumin and alkalisalts of ethylendiamintetracetic acid, among others.

Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(capry late/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.). JohnWiley and Sons, NY, pp 51-94 (1995) and Todd et al., Vaccine 15:564-570 (1997). (The teachings and content of which are hereby incorporated by reference.)

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book. (The teachings and content of which are hereby incorporated by reference.)

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with an allyl sucrose or with allyl pentaerythritol. Among then, there may be mentioned Carbopol 974P, 934P and 971P. Most preferred is the use of Cabopol 971P. Among the copolymers of maleic anhydride and alkenyl derivative, the copolymers EMA (Monsanto) which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide among many others.

Preferably, the adjuvant is added in an amount of about 100 μg to about 10 mg per dose. Even more preferably, the adjuvant is added in an amount of about 100 μg to about 10 mg per dose. Even more preferred the adjuvant is added in an amount of about 500 μg to about 5 mg per dose. Even more preferred the adjuvant is added in an amount of about 750 μg to about 2.5 mg per dose. Most preferred the adjuvant is added in an amount of about 1 mg per dose.

The vaccine composition can further include one or more other immunomodulatory agents such as, e.g. interleukins, interferons, or other cytokines. The vaccine compositions can also include Gentamicin and Merthiolate. While the amounts and concentrations of adjuvants and additives useful in the context of the present invention can readily be determined by the skilled artisan, the present invention contemplates compositions comprising from about 20 μg to about 2000 μg of adjuvant and preferably about 250 μg/ml dose of the vaccine composition. In another preferred embodiment, the present invention contemplates vaccine compositions comprising from about 1 ug/ml to about 60 μg/ml of antibiotics, and more preferably less than about 30 μg/ml of antibiotics.

With exception of any subunit vaccine, the vaccine is preferably administered to the horses in any conventional manner, most preferably through orally via oral drench, milk- and/or water drinking. The dosage to be administered will depend upon the particular case, but in any event, it is the amount sufficient to induce a protective antibody or cell-mediated immune response against ileitis.

As described above, the vaccines according to the invention are generally administered to the horses, preferably at week one (1) of age or older, more preferably between week one (1) and twelve (12), preferably one (1) and ten (10) of age, even more preferably between week one (1) and eight (8) of age, even more preferably between week one (1) and seven (7) of age, even more preferably between week one (1) and six (6) of age, even more preferably between week one (1) and five (5) of age, and most preferably between week one (1) and four (4) of age. Preferably, the effective dose is that as described above, for example about 4.5 log 10 TCID₅₀ to about 6.9 log 10 TCID₅₀ when a live modified L. intracellularis bacteria is used, in particular if modified L. intracellularis bacteria is used that is included in the vaccine Enterisol® Ileitis or Enterisol® Ileitis B3903 (Boehringer Ingelheim Vetmedica, Inc.).

If a second administration is desirable or necessary, the second administration is performed about 1 to about 4 weeks after the first administration of the vaccine. According to a further aspect, revaccination is performed in an interval of 3 to 12 month after administration of any previous vaccination. Administration of subsequent vaccine doses is preferably done on an 6 month to an annual basis. In another preferred aspect, animals vaccinated before the age of about 2 to 3 weeks should be revaccinated. Administration of subsequent vaccine doses is preferably done on an annual basis.

The present invention is further described in the following examples which are provided for illustrative purposes only and are not to be construed as limiting. Indeed, other variants of the invention will be readily apparent to one of ordinary skill in the art.

All publications and patents cited herein are incorporated by reference in their entireties.

### EXAMPLES

### Example 1

### Use of Lawsonia intracellularis Antigen in Horses after Oral Application

### Objectives

The objective of this trial was to determine the safety of Lawsonia intracellularis B3903 (Enterisol® Ileitis) in horses after oral application. The clinical symptoms (diarrhea and fever) and clinical pathology (Hematology and serum chemistry) were examined and used to determine safety of the vaccine in a non-target species, equine. Shedding of the vaccine organism was also followed.

### Experimental Design

This study consisted of two treatment groups consisting of ten foals each. The age of the foals was 7 months ±2 weeks. All study animals were treated with either the test article or control article diluted into 300 milliliters of distilled water. The treatment was administered using a nasal-gastric tube. Clinical observations were made daily for the duration of the study.

### Results

TABLE 4
Summary of Clinical Observations and Clinical Chemistry and Hematology Test Results

| Criteria to Assess Safety | Results |
|---|---|
| Clinical observations | No clinical signs observed that could be |
| | attributed to the test/control article |
| Fever (Rectal Temperature) | No significant increase in rectal |
| | temperature following administration of |
| | test/control article |
| Detection of shedding of Lawsonia | No Lawsonia intracellularis was detected in |
| intracellularis using PCR | fecal samples prior to or following |
| | treatment |
| Hypoproteinemia | No significant decrease in serum protein |
| | level |
| Hyponatremia | No significant decrease in serum sodium |
| | level |
| Hypocloremia | No significant decrease in serum chloride |
| | level |
| Hypocalcaemia | No significant decrease in serum calcium |
| | level |
| Increase in Creatine Kinase | No significant increase is serum Creatine |
| | Kinase |
| Anemia (Cell count or Hematocrit) | No significant change in either RBC count |
| | or hematocrit |
| Neutrophil band cells | No increase in Neutrophil band cells |
| Segmented Neutrophils | No significant increase in segmented |
| | Neutrophils |
| Leucocytosis (WBC, White Blood Cells) | No significant increase in WBC numbers |
| Weight gain | No significant effect on weight gain |

### Discussion

This study consisted of two groups with ten foals each. The control group was treated with Water for Injection (WFI) and the test group was treated with an avirulent live culture of Lawsonia intracellularis. The volume of either the control article or test article was 20 milliliters diluted into 300 ml of distilled water and administered using a nasal-gastric tube. Following treatment, animals were monitored for observable clinical symptoms. Rectal temperature was measured prior to treatment, at 4 hours post treatment, and daily for four days following treatment. On the three days post treatment and weekly there after, whole blood was drawn to monitor for an increase in neutrophil segmented cells numbers, for the appearance of neutrophil band cells, the presence of leucocytosis and anemia. On the same schedule, blood for serum was collected to evaluate serum chemistry which included creatine kinase, hypocalcemia, hypocloremia, and hyponatremia. The foals were weighed on Trial Days—4 and 29, to evaluate the effect of the treatment on weight gain. On the three days post treatment and weekly there after, fecal samples were collected for testing by Polymerase Chain Reaction (PCR) specifically for the presence of Lawsonia intracellilaris. Finally, serological testing was performed on serum samples collected on Trial Day 0 and 29.

No significant differences were noted when comparing the serum albumin levels and total protein levels of the control foals with the test foals. Comparing the serum levels of chloride, creatine levels were likewise not significantly different for the two treatment groups. Significant differences between the treatment groups for calcium and sodium existed on trial dates 7 and 29, respectively. In each case, the levels were just outside the normal range Whole blood analysis resulted in no significant differences for any of the parameters tested, increase in neutrophil segmented cells, increase in neutrophil band cells, anemia (RBC counts or HCT), and increase of WBC counts. No clinically significant rectal temperatures were measured as defined by an increase of 2° C. for two consecutive days over the pretreatment baseline. No significant difference existed for the average daily weight gain between the two groups. All fecal samples tested negative for the presence of Lawsonia intracellularis by PCR. Also all foals tested negative for antibody to Lawsonia intracellularis on Trial Day 0 and +29.

During the entire study, all foals were observed to be normal or without clinical symptoms that could be a result of either treatment. The results of this study illustrate the safety in six to seven and half month old foals of a Lawsonia intracellularis vaccine, avirulent Live Culture that was administered orally.

1) A method for the prevention or treatment of a horse against L. intracellularis infections, for reduction of clinical symptoms caused by L. intracellularis in a horse, or for the prevention of sub-clinical effects, preferably subclinical enteritis caused by or associated with L. intracellularis infection in a horse, comprising the step administering to said horse an effective dose of L. intracellularis antigen.

2) The method according to claim 1, wherein the horse is vaccinated at 1 week of age or older.

3) The method according to claim 1, wherein the horse is vaccinated between week 1 and 8 of age.

4) The method according to claim 1, wherein the L. intracellularis antigen is selected from the group consisting of a live modified L. intracellularis bacteria, a killed L. intracellularis bacteria or one or more sub-units of L. intracellularis bacteria.

5) The method according to claim 1, wherein the L. intracellularis antigen is live modified L. intracellularis bacteria.

6) The method according to claim 5, wherein the horse is administered with a dose of about 4.9 log 10 to about 6.9 log 10 of the live modified L. intracellularis bacteria.

7) The method according to claim 1, wherein the clinical symptom is selected from the group consisting of chronic weight loss, unthriftiness, fever, diarrhea, colic, leucocytosis and/or hypoproteinaemia.

8) Use of an effective dose of L. intracellularis antigen for the preparation of a medicament for the prevention or treatment of a horse against L. intracellularis infections, for reduction of clinical symptoms caused by L. intracellularis in a horse or for the prevention of subclinical effects, preferably subclinical enteritis caused by or associated with L. intracellularis infection in a horse, wherein said effective dose of L. intracellularis antigen is administered to a horse in of such treatment.

9) The use according to claim 8, wherein the horse is vaccinated at 1 week of age or older.

10) The use according to claim 8, wherein the horse is vaccinated between week 1 and 8 of age.

11) The use according to claim 8, wherein the L. intracellularis antigen is selected from the group consisting of live modified L. intracellularis bacteria, killed L. intracellularis bacteria or one or more sub-units of L. intracellularis bacteria.

12) The use according to claim 8, wherein the L. intracellularis antigen is live modified L. intracellularis bacteria.

13) The use according to claim 12, wherein the horse is administered with a dose of about 4.9 log 10 to about 6.9 log 10 of the live modified L. intracellularis bacteria.

14) The use according to claim 8, wherein the clinical symptom is selected from the group consisting of: chronic weight loss, unthriftiness, fever, diarrhea, colic, leucocytosis and/or hypoproteinaemia.
